(19) United States
(12) Patent Application Publication
(10) Pub. No.: US 2020/0163993 A1
(43) Pub. Date: May 28, 2020

(54) TREATMENT OF CANCER AND INFECTIOUS DISEASES WITH NATURAL KILLER (NK) CELL-DERIVED EXOSOMES

(71) Applicants: Saint Louis University, St. Louis, MO (US); U.S. Department of Veterans Affairs, Washinhton, DC (US)

(72) Inventor: Jacki KORNBLUTH, Ballwin, MO (US)

(73) Assignees: Saint Louis University, St. Louis, MO (US); U.S. Department of Veterans Affairs, Washington, DC (US)

(21) Appl. No.: 16/611,664

(22) PCT Filed: May 8, 2018
(86) PCT No.: PCT/US2018/031468

§ 371 Date: Nov. 7, 2019

Related U.S. Application Data
(60) Provisional application No. 62/503,671, filed on May 9, 2017.

(51) Int. Cl. A61K 35/17; C12N 5/0783; A61K 45/06

(52) U.S. Cl. CPC A61K 35/17; C12N 5/0646; C12N 2501/999; C12N 2501/2302; A61K 45/06

(57) ABSTRACT

Disclosed are therapeutic compositions, methods of preparing, and methods use for the treatment of infectious diseases and cancer. More specifically, Natural Killer Cell (NK Cell)-derived exosomes are demonstrated as potent therapeutic agents that can kill tumor cells in vivo, but do not kill normal cells. The exosomes can also be used to treat infectious disease.

This application claims benefit of priority to U.S. Provisional Application Ser. No. 62/503,671, filed May 9, 2017, the entire contents of which are hereby incorporated by reference.

This invention was made with government support under VA Merit Award BX000705 awarded by the Veterans' Administration. The government has certain rights in the invention.

### BACKGROUND

### 1. Field

The present disclosure relates generally to the fields of medicine, immunology, infectious disease and oncology. More particularly, the disclosure provides methods and compositions for the production and use of NK-derived exosomes for the treatment of cancer and infectious disease.

### 2. Description of Related Art

Exosomes are nanovesicles naturally released by almost all cells. In both normal and diseased states, exosomes deliver various molecules, such as proteins, lipids and nucleic acids, to target cells. Given their ability to interact with the surface of target cells, including ligand-receptor interactions and plasma membrane fusion, the transfer of exosome content to the target cell cytoplasm can be achieved.

Human natural killer (NK) cells have been demonstrated to release exosomes in both resting and activated condition (Lugini et al., J. Immunol. 189:2833-42, 2012). NK cell-derived exosomes have been shown to not only express typical NK markers (e.g., CD56) and killer proteins (e.g., FASL and perforin), but to exert antitumor and immune homeostatic activities. These findings suggest that NK cells secrete exosomes in a constitutive fashion, independent from their activation status, which may in turn suggest that NK cell-derived exosomes can control the immune response without specific stimuli. In fact, resting NK cell-derived exosomes contain both FASL and perforin. Together with CD56, FASL and perforin, NK cell-derived exosomes also express detectable amounts of the activating receptor NKG2D. Collectively, the properties of NK cell exosomes suggest an intriguing potential in the treatment of diseases such as cancer and infection.

### SUMMARY

Thus, in accordance with the present disclosure, there is provided a method of preparing an NK cell exosome composition comprising (a) culturing an NK cell is the presence of IL-2, phorbol ester PMA and calcium ionophore; and (b) collecting the exosomes produced by the NK cell of step (a) using precipitation or ultracentrifugation. The method may further comprise assessing the collected exosomes for the presence of one or more of granzyme B, perforin, NKLAM, CD63 and/or LAMP-1. Precipitation may comprise polymer-mediated precipitation (e.g., ExoQuick®) to isolate exosomes from the supernatant of NK cells cultured in the presence of IL-2, PMA and calcium ionophore. Ultracentrifugation may comprise a series of differential centrifugations to enrich exosomes from the supernatant of NK cells cultured in the presence of IL-2, PMA and calcium ionophore. Culturing may comprise (a) stimulating NK cells with IL-2 or other activating cytokine, followed by (b) stimulation with PMA and calcium ionophore, optionally wherein (a) is about 12-18 hours in duration and (b) is about 4-6 hours in duration. The method may also further comprise purifying said exosomes by polymer-based precipitation or differential ultracentrifugation. The NK cell may be a human NK cell or a non-human mammalian NK cell. The method may further comprise freezing the collected exosomes. The amount of NK cell exosomes produced per 10⁶ NK cells may be about 5 μg to about 20 μg.

In another embodiment, there is provided a method of treating a subject with cancer comprising administering to said subject an NK cell exosome preparation prepared according to the methods above. The method may further comprising administering to said subject a second anti-cancer therapy, such as chemotherapy, radiotherapy, immunotherapy, hormonal therapy, a toxin therapy or surgery. The cancer may be lung cancer, head and neck cancer, breast cancer, pancreatic cancer, prostate cancer, thyroid cancer, brain cancer, renal cancer, bone cancer, liver cancer, skin cancers including melanoma, testicular cancer, cervical cancer, ovarian cancer gastrointestinal cancer, leukemia, lymphomas, colon cancer, or bladder cancer. The NK cell exosome preparation may be administered more than once, such as on a chronic basis. The NK cell exosome preparation may be administered systemically, or administered intratumorally, or local or regional to a tumor. The cancer may be metastatic, recurrent and/or multi-drug resistant. The NK cell exosome preparation may be prepared using an NK cell from the subject, a healthy donor, umbilical cord blood or a NK cell line, including but not restricted to NK92 and NK3.3.

In still another embodiment, there is provided a method of treating a subject with an infectious disease comprising administering to said subject an NK cell exosome preparation prepared by the method set out above. The method of may further comprise administering to said subject a second infectious disease therapy. The infectious disease may be a bacterium, and said second infectious disease therapy is an antibiotic. The infectious disease may be a virus, and said second infectious disease therapy is an antiviral. The NK cell exosome preparation is administered more than once, such as on a chronic basis. The NK cell exosome preparation may be administered systemically, or administered local or regional to a site of infection. The infectious disease may be drug resistant. The NK cell exosome preparation may be prepared using an NK cell from the subject, a healthy donor, umbilical cord blood or a NK cell line.

Other objects and features of the present disclosure will become apparent from the following detailed description. It should be understood, however, that the description and the specific examples, while indicating particular embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Immunoblot analysis of exosomes from IL-2+P+I treated NK3.3 cells. Exosomes contain exosome specific proteins CD9, CD63 and TSG101 and NK specific proteins Natural Killer Lytic-Associated Molecule NKLAM, Fas ligand (FasL), granulysin, perforin, granzyme B and LAMP-1.

FIG. 2. NK3.3 derived exosomes inhibit K562 but not normal lymphocytes. K562 and cord blood lymphocytes (CB) were treated with exosomes from IL-2+P+I stimulated NK3.3 cells. Luminescence measures cell metabolic activity over time.

FIG. 3. Protein lysates from K562 cells untreated (−) or treated with exosomes from IL-2+P+I-stimulated NK3.3 cells (+) for 18 hr immunoblotted for myc, Bcl-2 and UCKL-1. β-actin was the loading control.

FIG. 4. Fresh or frozen and thawed NK3.3-derived exosomes inhibit K562. K562 cells were treated with varying concentrations (0.5, 1.0 and 10 μg) of fresh or frozen and thawed NK3.3 derived exosomes. Luminescence measures cell metabolic activity over time. Both fresh and frozen and thawed exosomes have equivalent tumor growth inhibitory properties.

FIG. 5. Immunodeficient NSG (NOD/SCID IL-2Rγ−/−) mice were injected subcutaneously in the right flank with human tumor cells K562 (1 million cells/mouse) in a gel matrix (matrigel). When tumors became palpable (day 8), they were injected intratumorally with NK3.3 derived exosomes or PBS (as a negative control). Mice were injected with 5 μg of exosomes. After 3 more days, tumor-bearing mice were injected again with exosomes (15 μg/mouse) or PBS (day 11). A final intratumoral injection of 15 μg exosomes/mouse was given on day 13. Mice were sacrificed on day 15, tumors were excised, formalin-fixed, and paraffin embedded. Sections of tumor were made, placed on slides and stained with hematoxylin and eosin to evaluate histology. The top panel represents tumors from control-treated mice. The tumor cells are healthy, intact and proliferating. In contrast, the bottom panel represents tumors from exosome-treated mice. These have large areas of apoptotic/dead tumor cells.

### DESCRIPTION OF ILLUSTRATIVE EMBODIMENTS

As discussed above, Natural Killer (NK) cells are a small subpopulation of circulating white blood cells that act as the first line of defense in the body's response to tumors and infectious agents. The inventor previously developed one of the only human NK cell lines, called NK3.3. These cells originated from the peripheral blood of a normal adult male and have all of the characteristics of a normal NK cell. It was recently found that NK cells can release small membrane-bound vesicles, called exosomes, which can kill tumor cells in vitro, but do not kill normal cells. Exosomes are naturally-occurring nanoparticles containing proteins, lipids and RNA that can transfer information. Using NK3.3 cells, the inventor has optimized a protocol for generating and purifying exosomes with the ability to kill diseased cells like tumor cells and virus-infected cells. These exosomes, unlike NK cells, are very stable. The inventor found that both fresh and frozen exosomes from NK3.3 cells work equally well, and can be produced in large quantities. NK3.3-derived exosomes have the potential to be an “off-the-shelf” product for treatment of cancer and viral infections.

Thus, NK exosomes constitute a new treatment for cancer as well as viral diseases where NK cells play a role, including herpesviruses, HIV, hepatitis viruses, measles, cytomegalovirus, influenza viruses, and pox viruses. The advantage of exosomes over cell-based therapies is that exosomes are stable, can be prepared in large batches, frozen, and stored indefinitely. Moreover, they maintain their activity upon thawing, which means they can be administered immediately. And since they are not restricted by blood group or histocompatibility antigens, they can be given to anyone without the need for typing or cross matching, greatly enhancing their therapeutic application.

These and other aspects of the disclosure are presented in detail below.

### I. DEFINITIONS

The phrases “isolated” or “biologically pure” refer to material which is substantially or essentially free from components which normally accompany the material as it is found in its native state. Thus, isolated peptides in accordance with the disclosure preferably do not contain materials normally associated with the peptides in their in situ environment.

“Abnormal cell” is any cell that is considered to have a characteristic that is atypical for that cell type, including atypical growth, typical growth in an atypical location or typical action against an atypical target. Such cells include cancer cells, benign hyperplastic or dysplastic cells, inflammatory cells or autoimmune cells.

As used herein the specification, “a” or “an” may mean one, or more than one, or at least one. As used herein “another” may mean at least a second or more.

The term “exosomes,” as used herein, refers to a membranous particle having a diameter (or largest dimension where the particles is not spheroid) of between about 10 nm to about 5000 nm, more typically between 30 nm and 1000 nm, and most typically between about 50 nm and 200 nm, wherein at least part of the membrane of the exosomes is directly obtained from a cell membrane. Most commonly, exosomes will have a size (average diameter) that is up to 5% of the size of the donor cell. Therefore, especially contemplated exosomes include those that are shed from a cell. Platelets or their secreted particles are specifically excluded from this definition of exosomes.

As used herein, the term “sample” refers to any sample suitable for the methods provided by the present embodiments. The sample may be any sample that includes exosomes suitable for detection or isolation. Sources of samples include blood, bone marrow, pleural fluid, peritoneal fluid, cerebrospinal fluid, urine, saliva, amniotic fluid, ascites, broncho-alveolar lavage fluid, synovial fluid, breast milk, sweat, tears, joint fluid, and bronchial washes. In one aspect, the sample is a blood sample, including, for example, whole blood or any fraction or component thereof including serum and plasma. A blood sample suitable for use with the present disclosure may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer. In another embodiment, the sample may be platelet-free plasma.

### II. NK CELLS

Natural killer cells or NK cells are a type of cytotoxic lymphocyte critical to the innate immune system. The role NK cells play is analogous to that of cytotoxic T cells in the vertebrate adaptive immune response. NK cells provide rapid responses to viral-infected cells, acting at around 3 days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. This role is especially important because harmful cells that are missing MHC I markers cannot be detected and destroyed by other immune cells, such as T lymphocyte cells.

NK cells (belonging to the group of innate lymphoid cells) are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter into the circulation. NK cells differ from natural killer T cells (NKTs) phenotypically, by origin and by respective effector functions; often, NKT cell activity promotes NK cell activity by secreting IFNγ. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. The NKp46 cell surface marker constitutes, at the moment, another NK cell marker of preference being expressed in both humans, several strains of mice (including BALB/c mice) and in three common monkey species.

In addition to the knowledge that natural killer cells are effectors of innate immunity, recent research has uncovered information on both activating and inhibitory NK cell receptors which play important function roles including self-tolerance and sustaining NK cell activity. NK cells also play a role in adaptive immune response, numerous experiments have worked to demonstrate their ability to readily adjust to the immediate environment and formulate antigen-specific immunological memory, fundamental for responding to secondary infections with the same antigen. The role of NK cells in both the innate and adaptive immune responses is becoming increasingly important in research using NK cell activity and potential cancer therapies.

NK cell receptors can also be differentiated based on function. NK cells are not a subset of the T lymphocyte family. Natural cytotoxicity receptors directly induce apoptosis after binding to Fas ligand that directly indicate infection of a cell. The MHC-dependent receptors (described above) use an alternate pathway to induce apoptosis in infected cells. Natural killer cell activation is determined by the balance of inhibitory and activating receptor stimulation. For example, if the inhibitory receptor signaling is more prominent, then NK cell activity will be inhibited; similarly, if the activating signal is dominant, then NK cell activation will result.

NK cell receptor types (with inhibitory, as well as some activating members) are differentiated by structure, with a few examples to follow:
Ly49 (homodimers), relatively ancient, C-type lectin family receptors, are of multigenic presence in mice, while humans have only one pseudogenic Ly49, the receptor for classical (polymorphic) MHC I molecules.
NCR (natural cytotoxicity receptors), upon stimulation, mediate NK killing and release of IFNγ.
CD94: NKG2 (heterodimers), a C-type lectin family receptor, is conserved in both rodents and primates and identifies nonclassical (also nonpolymorphic) MHC I molecules such as HLA-E. Expression of HLA-E at the cell surface is dependent on the presence of nonamer peptide epitope derived from the signal sequence of classical MHC class I molecules, which is generated by the sequential action of signal peptide peptidase and the proteasome. Though indirect, this is a way to survey the levels of classical (polymorphic) HLA molecules.
CD16 (FcγIIIA) plays a role in antibody-dependent cell-mediated cytotoxicity; in particular, they bind IgG.
Killer-cell immunoglobulin-like receptors (KIRs) belong to a multigene family of more recently evolved Ig-like extracellular domain receptors; they are present in nonhuman primates, and are the main receptors for both classical MHC I (HLA-A, HLA-B, HLA-C) and nonclassical Mamu-G (HLA-G) in primates. Some KIRs are specific for certain HLA subtypes. Most KIRs are inhibitory and dominant. Regular cells express MHC class 1, so are recognised by KIR receptors and NK cell killing is inhibited.
ILT or LIR (leukocyte inhibitory receptors) are recently discovered members of the Ig receptor family.
Ly49 (homodimers) have both activating and inhibitory isoforms. They are highly polymorphic on the population level; though they are structurally unrelated to KIRs, they are the functional homologues of KIRs in mice, including the expression pattern. Ly49s are receptor for classical (polymorphic) MHC I molecules.
   NK cells are cytotoxic; small granules in their cytoplasm contain proteins such as perforin and proteases known as granzymes. Upon release in close proximity to a cell slated for killing, perforin forms pores in the cell membrane of the target cell, creating an aqueous channel through which the granzymes and associated molecules can enter, inducing either apoptosis or osmotic cell lysis. The distinction between apoptosis and cell lysis is important in immunology: lysing a virus-infected cell could potentially only release the virions, whereas apoptosis leads to destruction of the virus inside. α-defensins, antimicrobial molecules, are also secreted by NK cells, and directly kill bacteria by disrupting their cell walls in a manner analogous to that of neutrophils.

Infected cells are routinely opsonized with antibodies for detection by immune cells. Antibodies that bind to antigens can be recognised by FcγRIII (CD16) receptors expressed on NK cells, resulting in NK activation, release of cytolytic granules and consequent cell apoptosis. This is a major killing mechanism of some monoclonal antibodies like rituximab (Rituxan), ofatumumab (Azzera), and others. The contribution of antibody-dependent cell-mediated cytotoxicity to tumor cell killing can be measured with a specific test that uses NK-92 that has been transfected with a high-affinity FcR. Results are compared to the “wild type” NK-92 that does not express the FcR. NK3.3 cells express FcR and can be used to measure antibody-dependent cellular cytotoxicity by evaluating tumor killing in the presence and absence of antibody.

Cytokines play a crucial role in NK cell activation. As these are stress molecules released by cells upon viral infection, they serve to signal to the NK cell the presence of viral pathogens in the affected area. Cytokines involved in NK activation include IL-12, IL-15, IL-18, IL-2, and CCL5. NK cells are activated in response to interferons or macrophage-derived cytokines. They serve to contain viral infections while the adaptive immune response generates antigen-specific cytotoxic T cells that can clear the infection. NK cells work to control viral infections by secreting IFNγ and TNFα. IFNγ activates macrophages for phagocytosis and lysis, and TNFα acts to promote direct NK tumor cell killing. Patients deficient in NK cells prove to be highly susceptible to early phases of herpes virus infection.

For NK cells to defend the body against viruses and other pathogens, they require mechanisms that enable the determination of whether a cell is infected or not. The exact mechanisms remain the subject of current investigation, but recognition of an “altered self” state is thought to be involved. To control their cytotoxic activity, NK cells possess two types of surface receptors: activating receptors and inhibitory receptors, including killer-cell immunoglobulin-like receptors. Most of these receptors are not unique to NK cells and can be present in some T cell subsets, as well.

These inhibitory receptors recognize MHC class I alleles, which could explain why NK cells preferentially kill cells that possess low levels of MHC class I molecules. This mode of NK cell target interaction is known as “missing-self recognition,” a term coined by Klas Kärre and co-workers in the late 1990's. MHC class I molecules are the main mechanism by which cells display viral or tumor antigens to cytotoxic T cells. A common evolutionary adaptation to this is seen in both intracellular microbes and tumors: the chronic down-regulation of MHC I molecules, which makes affected cells invisible to T cells, allowing them to evade T cell-mediated immunity. NK cells apparently evolved as an evolutionary response to this adaptation (the loss of the MHC eliminates CD4/CD8 action, so another immune cell evolved to fulfill the function).

Natural killer cells often lack antigen-specific cell surface receptors, so are part of innate immunity, i.e. able to react immediately with no prior exposure to the pathogen. In both mice and humans, NKs can be seen to play a role in tumor immunosurveillance by directly inducing the death of tumor cells (NKs act as cytolytic effector lymphocytes), even in the absence of surface adhesion molecules and antigenic peptides. This role of NK cells is critical to immune success particularly because T cells are unable to recognize pathogens in the absence of surface antigens. Tumor cell detection results in activation of NK cells and consequent cytokine production and release.

If tumor cells do not cause inflammation, they will also be regarded as self and will not induce a T cell response. A number of cytokines are produced by NKs, including tumor necrosis factor α (TNFα), IFNγ, and interleukin (IL-10). TNFα and IL-10 act as proinflammatory and immunosuppressors, respectively. The activation of NK cells and subsequent production of cytolytic effector cells impacts macrophages, dendritic cells, and neutrophils, which subsequently enables antigen-specific T and B cell responses. Instead of acting via antigen-specific receptors, lysis of tumor cells by NK cells is mediated by alternative receptors, including NKG2D, NKp44, NKp46, NKp30, and DNAM. NKG2D is a disulfide-linked homodimer which recognizes a number of ligands, including ULBP and MICA, which are typically expressed on tumor cells.

NK cells, along with macrophages and several other cell types, express the Fc receptor (FcR) molecule (FC-gamma-RIII=CD16), an activating biochemical receptor that binds the Fc portion of antibodies. This allows NK cells to target cells against which a humoral response has been mobilized and to lyse cells through ADCC. This response depends on the affinity of the Fc receptor expressed on NK cells, which can have high, intermediate, and low affinity for the Fc portion of the antibody or IgG. This affinity is determined by the nucleotide status in position 158 of the gene, which can code phenylalanine (F allele) or valine (V allele). Individuals with high-affinity FcRgammRIII (158 V/V allele) respond better to antibody therapy. This has been shown for lymphoma patients who received the antibody Rituxan. Patients who express the 158 V/V allele had a better antitumor response. Only 15-25% of the population expressed the 158 V/V allele. To determine the ADCC contribution of monoclonal antibodies, NK-92 cells (a “pure” NK cell line) has been transfected with the gene for the high-affinity FcR. The human NK cell line NK3.3 naturally expresses the Fc receptor CD16 and is also used as a readout for ADCC.

The ability to generate memory cells following a primary infection and the consequent rapid immune activation and response to succeeding infections by the same antigen is fundamental to the role T and B cells play in the adaptive immune response. For many years, NK cells have been considered to be a part of the innate immune system. However, recently increasing evidence suggests that NK cells can display several features that are usually attributed to adaptive immune cells (e.g., T cell responses) such as expansion and contraction of subsets, increased longevity and a form of immunological memory, characterized by a more potent response upon secondary challenge with the same antigen. These exciting new data have been generated in a diverse set of experimental systems. In mice the majority of research was carried out with murine cytomegalovirus (MCMV) and in models of hapten-hypersensitivity reactions. In humans most studies focused on the expansion of an NK cell subset carrying the activating receptor NKG2C. This expansion was observed primarily in response to Human Cytomegalovirus (HCMV) but other infections, e.g., Hantavirus, have been reported to trigger expansion of NKG2C+ NK cells as well.

As the majority of pregnancies involve two parents who are not tissue-matched, successful pregnancy requires the mother's immune system to be suppressed. NK cells are thought to be an important cell type in this process. These cells are known as “uterine NK cells” (uNK cells) and they differ from peripheral NK cells. They are in the CD56^{bright} NK cell subset, potent at cytokine secretion, but with low cytotoxic ability and relatively similar to peripheral CD56^{bright} NK cells, with a slightly different receptor profile. These uNK cells are the most abundant leukocytes present in utero in early pregnancy, representing about 70% of leukocytes here, but from where they originate remains controversial.

These NK cells have the ability to elicit cell cytotoxicity in vitro, but at a lower level than peripheral NK cells, despite containing perforin. Lack of cytotoxicity in vivo may be due to the presence of ligands for their inhibitory receptors. Trophoblast cells downregulate HLA-A and HLA-B to defend against cytotoxic T cell-mediated death. This would normally trigger NK cells by missing self recognition; however, these cells survive. The selective retention of HLA-E (which is a ligand for NK cell inhibitory receptor NKG2A) and HLA-G (which is a ligand for NK cell inhibitory receptor KIR2DL4) by the trophoblast is thought to defend it against NK cell-mediated death.

Uterine NK cells have shown no significant difference in women with recurrent miscarriage compared with controls. However, higher peripheral NK cell percentages occur in women with recurrent miscarriages than in control groups.

NK cells secrete a high level of cytokines which help mediate their function. Some important cytokines they secrete include TNF-α, IL-10, IFN-γ, and TGF-β, among others. For example, IFN-γ dilates and thins the walls of maternal spiral arteries to enhance blood flow to the implantation site.

By shedding decoy NKG2D soluble ligands, tumor cells may avoid immune responses. These soluble NKG2D ligands bind to NK cell NKG2D receptors, activating a false NK response and consequently creating competition for the receptor site. This method of evasion occurs in prostate cancer. In addition, prostate cancer tumors can evade CD8 cell recognition due to their ability to downregulate expression of MHC class 1 molecules. This example of immune evasion actually highlights NK cells' importance in tumor surveillance and response, as CD8 cells can consequently only act on tumor cells in response to NK-initiated cytokine production (adaptive immune response).

In early experiments on cell-mediated cytotoxicity against tumor target cells, both in cancer patients and animal models, investigators consistently observed what was termed a “natural” reactivity; that is, a certain population of cells seemed to be able to lyse tumor cells without having been previously sensitized to them. As these discoveries were inconsistent with the established model at the time, many initially considered these observations to be artifacts. However, by 1973, ‘natural killing’ activity was established across a wide variety of species, and the existence of a separate lineage of cells possessing this ability was postulated.

Since NK cells recognize target cells when they express nonself HLA antigens (but not self), autologous (patients' own) NK cell infusions have not shown any antitumor effects. Instead, investigators are working on using allogeneic cells from peripheral blood, which requires that all T cells be removed before infusion into the patients to remove the risk of graft versus host disease, which can be fatal. This can be achieved using an immunomagnetic column (CliniMACS). In addition, because of the limited number of NK cells in blood (only 10% of lymphocytes are NK cells), their number needs to be expanded in culture. This can take a few weeks and the yield is donor-dependent. A simpler way to obtain high numbers of pure NK cells is to expand NK-92 cells whose cells continuously grow in culture and can be expanded to clinical grade numbers in bags or bioreactors. Clinical studies have shown it to be well tolerated and some antitumor responses have been seen in patients with lung cancer, melanoma, and lymphoma.

Infusions of T cells engineered to express a chimeric antigen receptor that recognizes an antigen molecule on leukemia cells could induce remissions in patients with advanced leukemia. Logistical challenges are present for expanding T cells and investigators are working on applying the same technology to peripheral blood NK cells and NK-92.

In a study at Boston Children's Hospital, in coordination with Dana-Farber Cancer Institute, whereby immunocompromised mice had contracted lymphomas from EBV infection, an NK-activating receptor called NKG2D was fused with a stimulatory Fc portion of the EBV antibody. The NKG2D-Fc fusion proved capable of reducing tumor growth and prolonging survival of the recipients. In a transplantation model of LMP 1-fueled lymphomas, the NKG2D-Fc fusion proved capable of reducing tumor growth and prolonging survival of the recipients.

Recent research suggests specific KIR-MHC class 1 gene interactions might control innate genetic resistance to certain viral infections, including HIV and its consequent development of AIDS. Certain HLA allotypes have been found to determine the progression of HIV to AIDS; an example is the HLA-B57 and HLA-B27 alleles, which have been found to delay progression from HIV to AIDS. This is evident because patients expressing these HLA alleles are observed to have lower viral loads and a more gradual decline in CD4⁺ T cells numbers. Despite considerable research and data collected measuring the genetic correlation of HLA alleles and KIR allotypes, a firm conclusion has not yet been drawn as to what combination provides decreased HIV and AIDS susceptibility.

NK cells can impose immune pressure on HIV, which had previously been described only for T cells and antibodies. HIV mutates to avoid NK cell activity. Human NK cells represent only about 2-4% of the lymphocytes in the blood, making it difficult to obtain large numbers of purified cells for study without great effort and expense. Nonetheless, primary human NK cells have been tested and produce useful exosomes.

NK 3.3 is a human natural killer (NK) cell line that was generated by the inventor in 1981. A description of the cells was first published in 1982 (J. Immunol. 129:2831-2837, 1982). These cells have been used by multiple investigators throughout the world. NK 3.3 is an important tool because the cells can be grown in the laboratory, providing a constant, ready supply of cells, and very large numbers of cells can be obtained. They are also derived from a single NK cell from a single individual, and therefore every NK 3.3 is identical to the next. It would be difficult and time consuming to obtain large numbers of NK cells from a single individual. In addition, not every NK cell from the same individual is the same. NK3.3 expresses CD2, CD56, CD16, NKp30, NKp46, NKG2D, CD161, CD122 on the cell surface and contains perforin, granzyme B, FASL, granulysin and NK-lytic associated molecule (NKLAM).

There is no other “normal” human NK cell line available except NK 3.3. There are at least two other human NK cell lines developed by other investigators, NK-92 and NKL. Both were derived from patients with leukemia. In contrast, NK 3.3 was generated from the blood of a healthy adult male. Another important feature of NK 3.3 is that it looks and acts the same as NK cells isolated from adult blood, and its activity can be regulated by the same factors that regulate normal NK cells, including cytokines such as IL-2, IL-12, IFN, IL-15. NK-92 and NKL do not behave like normal NK cells and their activity is not easily regulated.

NK 3.3 is thus only NK cell line that exhibits antibody-dependent cellular cytotoxicity, or ADCC. This is due to its cell surface expression of FcγRIII (CD16), which is not found on either NK-92 or NKL. Therefore NK 3.3 has potential important clinical utility in combination with antibody therapy for various diseases.

### III. EXOSOMES

A. Overview

The last decade has seen an exponential growth in the number of studies and publications related to extracellular microvesicles such as exosomes. These studies range from methods for their isolation to the role of certain extracellular microvesicles, particularly exosomes, in cancer and their ability to mediate immune responses. Release of extracellular microvesicles occurs in both prokaryotes and eukaryotes and is important in a broad range of physiological and pathological processes.

Extracellular microvesicles are cell-derived and cell-secreted microvesicles which, as a class, include exosomes, exosome-like vesicles, ectosomes (which result from budding of vesicles directly from the plasma membrane), microparticles, microvesicles, shedding microvesicles (SMVs), nanoparticles and even (large) apoptotic blebs or bodies (resulting from cell death) or membrane particles, because such terms have been used interchangeably in the field (Gyorgy et al., 2011; Simpson & Mathivanan, 2012).

“Extracellular microvesicles,” as used herein, include extracellular microvesicles referred to by terminologies used for naming in the past, including terms based on the sample source from which the extracellular microvesicles were derived. As applied to tumor exosomes in particular, the terms texosomes (tex) and oncosomes have been used and are included herein, as well as terms that reflect the particular type of cancer cell, such as prostate cancer cell-derived exosomes being termed prostasomes. In addition, exosomes isolated from dendritic cells have been termed dexosomes, and other nomenclatures have been used, such as epididimosomes, argosomes, promininosomes, prostasomes and archeosomes (Simpson & Mathivanan, 2012).

Although older terminologies are included herein, it is nonetheless advantageous to define “extracellular microvesicles” using more standardized nomenclature. Naming of extracellular microvesicles considers three known mechanisms by which membrane vesicles are released into the extracellular microenvironment: exocytic fusion of multivesicular bodies, resulting in “exosomes”; budding of vesicles directly from the plasma membrane, resulting in “ectosomes”; and cell death, leading to “apoptotic blebs.”

As used herein, the terms “microvesicles” and “MVs” typically mean larger extracellular membrane vesicles or structures surrounded by a phospholipid bilayer that are about 100 nm to about 1,000 nm in diameter, or about 100 nm to about 400 nm in blood plasma. Microvesicles/MVs are formed by regulated release by budding or blebbing of the plasma membrane.

“Exosome-like vesicles,” which have a common origin with exosomes, are typically described as having size and sedimentation properties that distinguish them from exosomes and, particularly, as lacking lipid raft microdomains. “Ectosomes”, as used herein, are typically neutrophil- or monocyte-derived microvesicles.

“Membrane particles” (MPs), as used herein, are typically about 50-80 nm in diameter and originate from the plasma membrane. “Extracellular membranous structures” also include linear or folded membrane fragments, e.g., from necrotic death, as well as membranous structures from other cellular sources, including secreted lysosomes and nanotubes.

As used herein, “apoptotic blebs or bodies” are typically about 1 to 5 μm in diameter and are released as blebs of cells undergoing apoptosis, i.e., diseased, unwanted and/or aberrant cells. They are characterized by PS externalization and may contain fragmented DNA.

Within the class of extracellular microvesicles, important components are “exosomes” themselves, which may be between about 40 to 50 nm and about 200 nm in diameter and being membranous vesicles, i.e., vesicles surrounded by a phospholipid bilayer, of endocytic origin, which result from exocytic fusion, or “exocytosis” of multivesicular bodies (MVBs) (Gyorgy et al., 2011; Simpson & Mathivanan, 2012). Less common, but included terms are also “vesiculation” and “trogocytosis”. In some cases, exosomes can be between about 40 to 50 nm up to about 200 nm in diameter, such as being from 60 nm to 180 nm.

Exosomes exert a broad array of important physiological functions, e.g., by acting as molecular messengers that traffic information between different cell types. For example, exosomes deliver proteins, lipids and soluble factors including RNA and microRNAs (Thery et al., 2009) which, depending on their source, participate in signaling pathways that can influence apoptosis (Andreola et al., 2002; Huber et al., 2005; Kim et al., 2005), metastasis (Parolini et al., 2009), angiogenesis (Kim et al., 2005; Iero et al., 2008), tumor progression (Keller et al., 2009; Thery et al., 2002), thrombosis (Aharon & Brenner, 2009; Nedawi et al., 2005) and immunity by directing T cells towards immune activation (Andre et al., 2004; Chaput et al., 2005) or immune suppression (Szajnik et al., 2010; Valenti et al., 2007; Wieckowski et al., 2009).

Exosomes incorporate a wide range of cytosolic and membrane components that reflect the properties of the parent cell. Therefore, the terminology applied to the originating cell can be used as a simple reference for the secreted exosomes. Accordingly, “NK-derived exosomes” can be used herein to indicate exosomes secreted by, derived from and indicative of, NK cells.

Because of the multiple intracellular fusion events involved in exosome formation, the luminal contents and proteomic and phospholipid profile of the extracellularly released vesicles mirrors that of the originating cell. The presence of cytosolic (nucleic acids) and plasma membrane constituents (proteins and phospholipids) from the originating cell provides a readily accessible surrogate that reflects the properties of the parent cell for biomarker analysis. For example, NK exosomes according to the present disclosure may contain 1, 2, 3, 4 or all 5 of NKLAM (exosome marker), CD63 (exosome marker), Granzyme B (cytotoxic lytic granule component), LAMP-1 (cytotoxic lytic granule component) and Perforin (cytotoxic lytic granule component).

B. Exosome Isolation

Some aspects of the embodiments concern isolation of exosomes. Exosomes may be isolated from freshly collected samples or from samples that have been stored frozen or refrigerated. Although not necessary, higher purity exosomes may be obtained if fluid samples are clarified before precipitation with a volume-excluding polymer, to remove any debris from the sample. Methods of clarification include centrifugation, ultracentrifugation, filtration, ultrafiltration and precipitation. Exosomes can be isolated by numerous methods well-known in the art. One method is differential centrifugation from body fluids. Exemplary methods for isolation of exosomes are described in Losche et al. (2004); Mesri & Altieri (1998); Morel et al. (2004) and International (PCT) Publication WO/2015/085096, each of which is incorporated herein by reference. Exosomes may also be isolated via flow cytometry as described in Combes et al. (1997), incorporated herein by reference.

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. For example, a commercial kit is available (EXOMIR™, Bioo Scientific) which allows removal of cells, platelets, and cellular debris on one microfilter and capturing of vesicles bigger than 30 nm on a second microfilter using positive pressure to drive the fluid. HPLC-based protocols could potentially allow one to obtain highly pure exosomes, though these processes require dedicated equipment and are difficult to scale up.

Similar techniques are described in the literature, including differential/ultracentrifugation (Thery et al., 2006); affinity chromatography (Taylor & Gercel-Taylor, 2008); polymer-mediated precipitation, particularly using polyethylene glycol (PEG) of different molecular weights, including the Total Exosome Isolation Reagents from Life Technologies Corporation (U.S. Pat. No. 8,901,284) and ExoQuick™ (U.S. Patent Publication 2013/0337440 A1); and capture on defined pore-size membranes such as ExoMir™, which typically uses two filters of different pore-sizes connected in series (U.S. Patent Publication 2013/0052647 A1).

A particular protocol is as follows. To prepare exosomes from NK3.3 cells with strong anti-tumor activity, NK3.3 cells are stimulated overnight in RPMI media containing 3% exosome-free fetal bovine serum (FBS) and 500 U/ml recombinant IL-2 at 5 million cells per ml. (The dose of IL-2 can range from 200 U/ml to 5000 U/ml and the time can range from 12-18 hours). Phorbol myristic acid (PMA) and A23187 calcium ionophore are then added to the cultures for an additional 5 hours. The dose of PMA is 80 ng/ml and A23187 is 4 μg/ml. (Doses can vary: PMA from 10-100 ng/ml and A23187 from 1-5 u/ml). Time can vary from 4-6 hours. After treatment, cells are pelleted at 3000×g for 15 minutes. The supernatant (containing exosomes) is transferred to a sterile tube. One method we use to prepare exosomes is to use the ExoQuick-TC Exosome Precipitation Solution. A ⅕ volume of ExoQuick is added, the sample is mixed and refrigerated overnight (at least 12 hours). The mixture is then centrifuged at 1500×g for 30 minutes. Exosomes are found in the pellet. They are resuspended in phosphate-buffered saline and either used immediately or aliquoted and stored at −80° C.

Stimulation of NK3.3 cells with an activating cytokine such as IL-2 overnight is critical to induce high levels of expression of the cytolytic molecules such as granzyme B, perforin and NKLAM. The PMA+A23187 treatment is critical to induce granule exocytosis, or degranulation. This leads to enhanced release of exosomes and vesicles with cytotoxic activity.

### IV. TREATMENT OF INFECTIOUS DISEASES

The present disclosure contemplates the treatment of infectious diseases, in particular viral disease, using NK cell exosomes. Viruses suitable for treatment include respiratory viruses such as Adenoviruses, Avian influenza, Influenza virus type A, Influenza virus type B, Measles, Parainfluenza virus, Respiratory syncytial virus (RSV), Rhinoviruses, and SARS-CoV, gastro-enteric viruses such as Coxsackie viruses, enteroviruses such as Poliovirus and Rotavirus, hepatitis viruses such as Hepatitis B virus, Hepatitis C virus, Bovine viral diarrhea virus (surrogate), herpesviruses such as Herpes simplex 1, Herpes simplex 2, Human cytomegalovirus, and Varicella zoster virus, retroviruses such as Human immunodeficiency virus 1 (HIV-1), and Human immunodeficiency virus 2 (HIV-2), as well as Dengue virus, Hantavirus, Hemorrhagic fever viruses, Lymphocytic choromeningitis virus, Smallpox virus, Ebola virus, Rabies virus, West Nile virus and Yellow fever virus.

It is envisioned that the NK cell exosomes described herein may be used in combination therapies with one or more additional therapies or agents that increase efficacy of either modality alone, and/or permit lower doses for effective treatment, thereby mitigating one or more of the side effects experienced by the patient. The following is a general discussion of therapies that may be used in conjunction with the therapies of the present disclosure.

In a combination therapy, one would administer to the patient disclosed NK cell exosomes and at least one other therapy. Both therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the NK cell exosome preparation and the other includes the other agent.

Alternatively, the NK cell exosomes described herein may precede or follow the other treatment by intervals ranging from minutes to weeks. One would generally ensure that a significant period of time did not expire between each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the NK cell exosome compositions or the other therapy will be desired. Various combinations may be employed, where an NK cell exosome preparation of the present disclosure is “A,” and the other therapy is “B,” as exemplified below:

A/B/A B/A/B B/A/A B/B/A A/A/B A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. The following is a general discussion of additional cancer therapies that may be used combination with the compositions of the present disclosure.

The term “antibiotics” are drugs which may be used to treat a bacterial infection through either inhibiting the growth of bacteria or killing bacteria. Without being bound by theory, it is believed that antibiotics can be classified into two major classes: bactericidal agents that kill bacteria or bacteriostatic agents that slow down or prevent the growth of bacteria.

The first commercially available antibiotic was released in the 1930's. Since then, many different antibiotics have been developed and widely prescribed. In 2010, on average, 4 in 5 Americans are prescribed antibiotics annually. Given the prevalence of antibiotics, bacteria have started to develop resistance to specific antibiotics and antibiotic mechanisms. Without being bound by theory, the use of antibiotics in combination with another antibiotic may modulate resistance and enhance the efficacy of one or both agents.

In some embodiments, antibiotics can fall into a wide range of classes. In some embodiments, the compounds of the present disclosure may be used in conjunction with another antibiotic. In some embodiments, the compounds may be used in conjunction with a narrow spectrum antibiotic which targets a specific bacteria type. In some non-limiting examples of bactericidal antibiotics include penicillin, cephalosporin, polymyxin, rifamycin, lipiarmycin, quinolones, and sulfonamides. In some non-limiting examples of bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the compounds could be combined with a drug which acts against mycobacteria such as cycloserine, capreomycin, ethionamide, rifampicin, rifabutin, rifapentine, and streptomycin. Other antibiotics that are contemplated for combination therapies may include arsphenamine, chloramphenicol, fosfomycin, fusidic acid, metronidazole, mupirocin, platensimycin, quinupristin, dalfopristin, thiamphenicol, tigecycline, tinidazole, or trimethoprim.

The term “antiviral” or “antiviral agents” are drugs which may be used to treat a viral infection. In general, antiviral agents act via two major mechanisms: preventing viral entry into the cell and inhibiting viral synthesis. Without being bound by theory, viral replication can be inhibited by using agents that mimic either the virus-associated proteins and thus block the cellular receptors or using agents that mimic the cellular receptors and thus block the virus-associated proteins. Furthermore, agents which cause an uncoating of the virus can also be used as antiviral agents.

The following are particular antiviral drugs that may be used in combination with the disclosed NK cell exosomes: Abacavir, Acyclovir, Adefovir, Amantadine, Amprenavir, Ampligen. Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Novir, Oseltamivir, Peginterferon alfa-2a, Penciclovir, Peramivir, Pleconaril, Podophyllotoxin, Raltegravir, Reverse transcriptase inhibitor, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir and Zidovudine.

### V. CANCER THERAPEUTIC METHODS

The present disclosure contemplates the treatment of hyperplastic/dysplastic/neoplastic diseases and conditions, including cancer. Types of diseases/conditions contemplated to be treated include lung cancer, head and neck cancer, breast cancer, pancreatic cancer, prostate cancer, thyroid cancer, brain cancer, renal cancer, bone cancer, liver cancer, skin cancers including melanoma, testicular cancer, cervical cancer, ovarian cancer gastrointestinal cancer, lymphomas, leukemia, colon cancer, bladder cancer and any other neoplastic diseases. Treatment will be understood to include killing cancer cells, inhibiting cell growth, inhibiting metastasis, decreasing tumor/tissue size, tumor cell burden or otherwise reversing or reducing the malignant phenotype of tumor cells. The routes of administration will vary, naturally, with the condition of the patient, type of cancer, location and nature of the lesion, and drug, and may include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation. Any of the formulations and routes of administration discussed with respect to the treatment or diagnosis of cancer may also be employed with respect to neoplastic diseases and conditions. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

It is envisioned that the NK cell exosomes described herein may be used in combination therapies with one or more additional therapies or agents that enhance the efficacy of either therapy alone, and/or that reduce dosing requirements such that one or more of the side effects experienced by the patient are mitigated. The following is a general discussion of therapies that may be used in conjunction with the therapies of the present disclosure.

To treat an cancer using the methods and compositions of the present disclosure, one would generally administer to the patient the composition and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the NK cell exosome preparation and the other includes the other agent.

Alternatively, the NK cell exosomes described herein may precede or follow the other treatment by intervals ranging from minutes to weeks. One would generally ensure that a significant period of time did not expire between each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the NK cell exosomes compositions or the other therapy will be desired. Various combinations may be employed, where an NK cell exosome preparation of the present disclosure is “A,” and the other therapy is “B,” as exemplified below:

A/B/A B/A/B B/A/A B/B/A A/A/B A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. The following is a general discussion of additional cancer therapies that may be used combination with the compositions of the present disclosure.

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1; dynemicin, including dynemicin A; uncialamycin and derivatives thereof; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein transferase inhibitors, transplatinum, 5-fluorouracil, vincristin, vinblastin and methotrexate and pharmaceutically acceptable salts, acids or derivatives of any of the above.

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believe to be particularly efficacious in reducing the reoccurrence of the tumor. Additionally, the compounds of the present disclosure can also be used in a neoadjuvant setting.

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-10, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents may be used in combination with the present disclosure to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VI. EXAMPLES

The following examples are included to demonstrate particular embodiments of the disclosure. It should be appreciated by those of skill in the art that the techniques disclosed in the examples which follow represent techniques discovered by the inventors to function well in the practice of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1—NK3.3-Derived Exosome Preparation

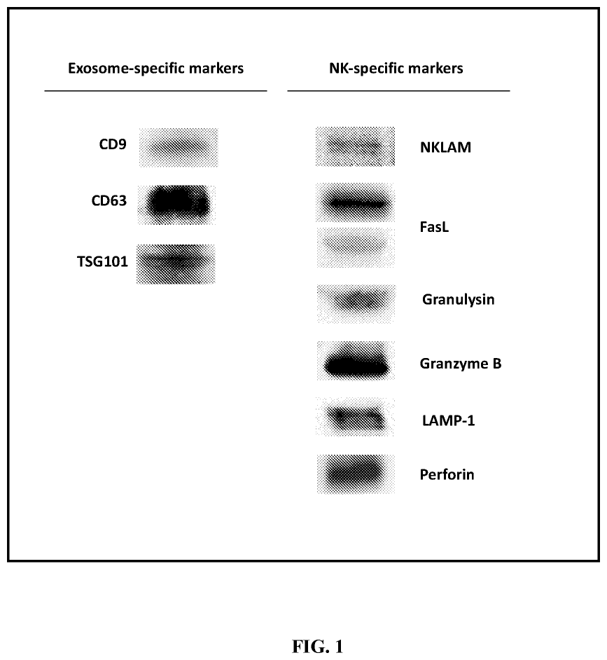

The inventor prepared exosomes from supernatants from interleukin-2 (IL-2)-stimulated NK3.3 cells after phorbol ester PMA and calcium ionophore (P+I) degranulation. Exosomes were prepared using the ExoQuick-TC® exosome precipitation solution and by ultracentrifugation, with similar results. The inventor verified the expression of cytotoxic lytic granule components granzyme B, perforin, LAMP-1 FASL, granulysin and NKLAM, and exosome markers CD63, CD9 and TSG101 (FIG. 1).

### Example 2—NK3.3-Derived Exosomes Enter Tumor Cells and Induce Cell Death

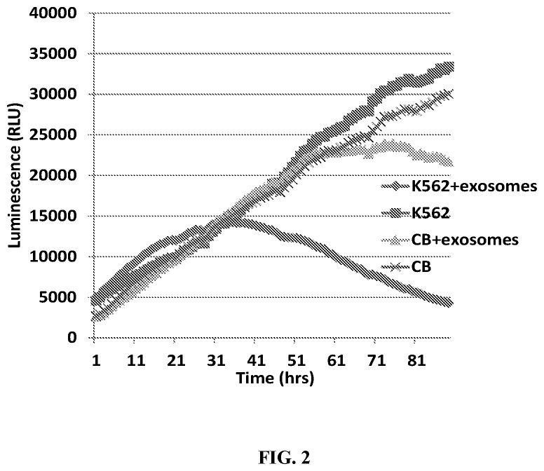

NK cells release cytolytically active exosomes. The inventors compared the ability of exosomes from IL-2 and IL-2+P+I stimulated NK3.3 cells to inhibit the growth of K562 tumor cells in vitro using a luminescence assay that measures metabolic activity (RealTime Glo®, Invitrogen). Exosomes from IL-2+P+I-treated NK cells are extremely effective in inhibiting K562 growth while exosomes from IL-2 stimulated NK3.3 cells are less effective. Exosomes from P+I-stimulated NK3.3 cells also contain higher levels of NKLAM. Importantly, the inventor found that NK-derived exosomes have a minimal effect on normal lymphocyte metabolism while strongly inhibiting K562 (FIG. 2). NKLAM-containing exosomes may therefore be a viable substitute for intact NK effector cells to selectively promote tumor cell death. These results are extremely exciting in that they open the door for NK-derived exosomes to be used in cancer therapy (“natural nanobullets”).

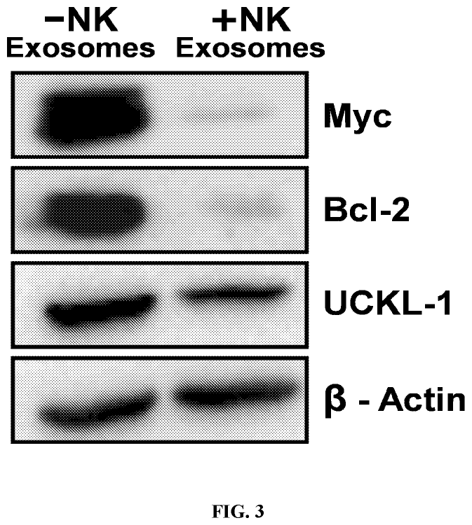

Upon exposure of K562 tumor cells to NKLAM-containing NK-derived exosomes, expression of the NKLAM substrate UCKL-1 is down-regulated. There is an even more dramatic decrease in myc and Bcl-2 levels (FIG. 3). These molecules control transcription and programmed cell death, important control mechanisms for cancer growth and metastasis.

### Example 3—In Vitro Exosome Experiments

The inventor has shown that in addition to human K562 tumor cells, NK3.3 derived exosomes kill a variety of other human hematopoietic tumor cells, including Raji (B cell), ARH77 (myeloma) and 8226 (myeloma). They do not kill normal human hematopoietic cells (cord blood lymphocytes; CB) or mouse tumor cells YAC-1.

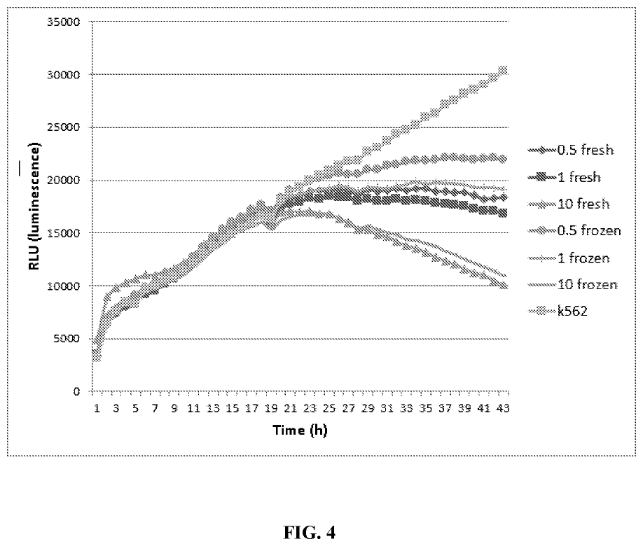

Exosomes have been reported to be extremely stable. The inventor prepared and froze aliquots of NK3.3-derived exosomes at −80° C. After two weeks, the frozen material was thawed to compare the ability of fresh and frozen material for anti-tumor activity. She found that both fresh and frozen exosomes had similar function. This indicates that large batches of exosomes can be prepared, frozen, stored and then thawed, without loss of function (FIG. 4).

### Example 4—In Vivo Exosome Experiments

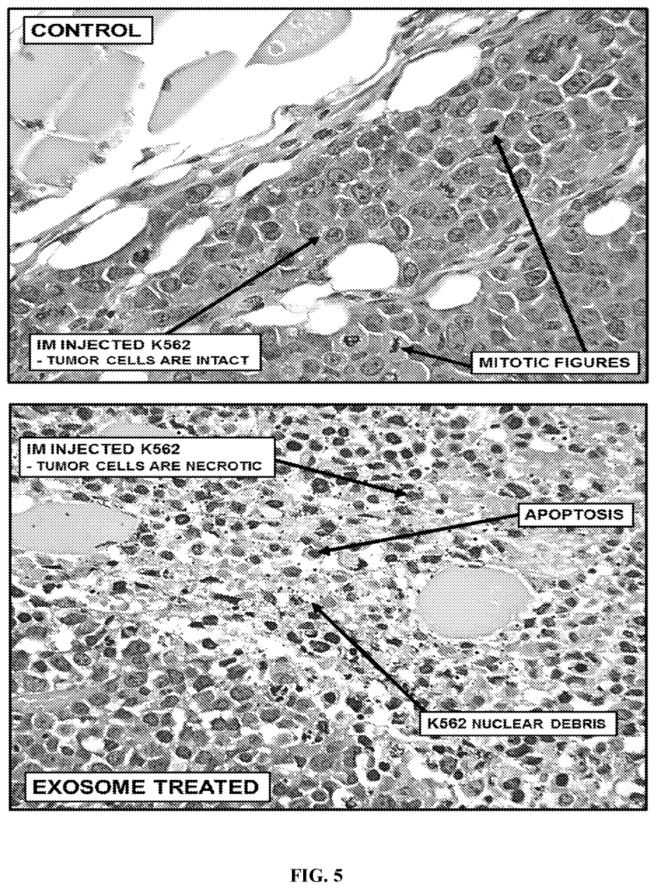

Immunodeficient NSG (NOD/SCID IL-2Rγ−/−) mice were injected subcutaneously in the right flank with human tumor cells K562 (1 million cells/mouse) in a gel matrix (matrigel). When tumors became palpable (day 8), they were injected intratumorally with NK3.3 derived exosomes or PBS (as a negative control). Mice were injected with 5 μg of exosomes. Tumor size was measured using calipers daily. Tumor volume was calculated as (width)²×length/2. After 3 more days, tumor-bearing mice were injected again with exosomes (15 gig/mouse) or PBS (day 11). A final intratumoral injection of 15 μg exosomes/mouse was given on day 13. Mice were sacrificed on day 15. Tumor growth was inhibited in 75% of animals treated with exosomes while tumors continued to grow in 100% of control-treated mice. To examine the tumors directly, tumors were excised, formalin-fixed, and paraffin embedded. Sections of tumor were made, placed on slides and stained with hematoxylin and eosin to evaluate histology. All of the tumors from exosome-treated mice had large areas of apoptotic/dead tumor cells while the tumor cells from PBS treated mice had minimal evidence of tumor death. These results indicate that NK3.3 derived exosomes inhibit tumor growth and are cytotoxic to human K562 tumor cells in vivo (FIG. 5).

All of the compositions and/or methods disclosed and claimed herein can be made and executed without undue experimentation in light of the present disclosure. While the compositions and methods of this disclosure have been described in terms of particular embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
U.S. Pat. No. 5,739,169
U.S. Pat. No. 5,801,005
U.S. Pat. No. 5,824,311
U.S. Pat. No. 5,830,880
U.S. Pat. No. 5,846,945
U.S. Pat. No. 8,901,284
U.S. 2013/0052647 A1
U.S. 2013/0337440 A1
WO/2015/085096
Aharon & Brenner, Best Practice & Research Clinical Haematology, 22:61-69, 2009.
Andre et al., J Immunol., 172:2126-2136, 2004.
Andreola et al., J Exp. Med., 195:1303-1316, 2002.
Austin-Ward and Villaseca, Rev. Med. Chil., 126(7):838-45, 1998.
Bukowski et al., Clin. Cancer Res., 4(10):2337-47, 1998.
Chaput et al., Expert. Opin. Biol. Ther., 5:737-747, 2005.
Christodoulides et al., Microbiology, 144(Pt 11):3027-37, 1998.
Combes et al., Thromb. Haemost., 77:220, 1997.
Davidson et al., J. Immunother., 21(5):389-98, 1998.
Gyorgy et al., Cell. Mo!. Life Sci., 68:2667-2688, 2011.
Hanibuchi et al., Int. J. Cancer, 78(4):480-485, 1998.
Hellstrand et al., Acta Oncol., 37(4):347-353, 1998.
Huber et al., Gastroenterology, 128:1796-1804, 2005.
Hui and Hashimoto, Infect. Immun., 66(11):5329-36, 1998.
Iero et al., Cell Death. Differ., 15:80-88, 2008.
Im et al., Nature Biotechnology, 32(5):490, 2014; doi: 10.1038/nbt.2886.
Keller et al., Cancer Lett., 278:73-81, 2009.
Kim et al., Clin. Cancer Res., 11:1010-1020, 2005.
Losche et al., Platelets, 15:109-115, 2004.
Mesri & Altieri, J. Immunol., 161:4382-4387, 1998.
Mitchell et al., Ann. NY Acad. Sci., 690:153-166, 1993.
Mitchell et al., J. Clin. Oncol., 8(5):856-869, 1990.
Mitchell et al., Proc. Natl. Acad. Sci. U.S.A 105, 10513-8. PM:18663219, 2008.
Morel et al., Curr. Opin. Hematol., 11:156-164, 2004.
Morton et al., Arch. Surg., 127:392-399, 1992.
Nedawi et al., Arterioscler. Thromb. Vase. Biol., 25:1744-1749, 2005.
Parolini et al., J. Biol. Chem., 284:34211-34222, 2009.
Pietras et al., Oncogene, 17(17):2235-49, 1998.
Qin et al., Proc. Natl. Acad. Sci. USA, 95(24):14411-14416, 1998.
Rabinowits et al., Clin. Lung Cancer 10, 42-6. PM:19289371, 2009.
Ravindranath and Morton, Intern. Rev. Immunol., 7: 303-329, 1991.
Rosenberg et al., Ann. Surg. 210(4):474-548, 1989.
Silva et al., Eur. Respir. J. 37, 617-23. PM:20595154, 2011.
Simpson & Mathivanan, J. Proteomics Bioinform., 5:2; 2012.
Smalley et al., J. Proteome. Res 7, 2088-96. PM:18373357, 2008.
Szajnik et al., PLoS One, 5:el 1469, 2010.
Taylor & Gercel-Taylor, Gynecol. Oncol. 110, 13-21. PM:18589210, 2008.
Thery et al., Curr. Protoc. Cell Biol., Chapter 3:Unit, 2006.
Thery et al., Nat. Rev. Immunol., 2:569-579, 2002.
Thery et al., Nat. Rev. Immunol., 9:581-593, 2009.
Thomas et al., Proteomes. 1, 87-108. PM:24860738, 2013.
Thorpe & Wawrzynczak, Cancer Res. 47(22):5924-31, 1987.
Valenti et al., Cancer Res., 67:2912-2915, 2007.
Wieckowski et al., J Immunol., 183:3720-3730, 2009.

1. A method of preparing an NK cell exosome composition comprising:
(a) culturing an NK cell is the presence of IL-2, phorbol ester PMA and calcium ionophore; and
(b) collecting the exosomes produced by the NK cell of step (a) using precipitation or ultracentrifugation.

2. The method of claim 1, further comprising assessing the collected exosomes for the presence of one or more of granzyme B, perforin, NKLAM, CD63 and/or LAMP-1.

3. The method of claim 1, wherein precipitation comprises polymer-mediated precipitation (e.g., ExoQuick®) to isolate exosomes from the supernatant of NK cells cultured in the presence of IL-2, PMA and calcium ionophore.

4. The method of claim 1, wherein ultracentrifugation comprises performing a series of differential centrifugations to enrich exosomes from the supernatant of NK cells cultured in the presence of IL-2, PMA and calcium ionophore.

5. The method of claim 1, wherein culturing comprises (a) stimulating NK cells with IL-2 or other activating cytokine, followed by (b) stimulation with PMA and calcium ionophore, optionally wherein (a) is about 12-18 hours in duration and (b) is about 4-6 hours in duration.

6. The method of claim 1, further comprising purifying said exosomes by polymer-based precipitation or differential ultracentrifugation.

7. The method of claim 1, wherein said NK cell is a human NK cell.

8. The method of claim 1, wherein said NK cell is a non-human mammalian NK cell.

9. The method of claim 1, further comprising freezing the collected exosomes.

10. The method of claim 1, wherein the amount of NK cell exosomes produced per 10⁶ NK cells is about 5 μg to about 20 μg.

11. A method of treating a subject with cancer comprising administering to said subject an NK cell exosome preparation prepared by the method of claim 1.

12. The method of claim 11, further comprising administering to said subject a second anti-cancer therapy.

13. (canceled)

14. The method of claim 11, wherein cancer is lung cancer, head and neck cancer, breast cancer, pancreatic cancer, prostate cancer, thyroid cancer, brain cancer, renal cancer, bone cancer, liver cancer, skin cancers including melanoma, testicular cancer, cervical cancer, ovarian cancer gastrointestinal cancer, leukemia, lymphomas, colon cancer, or bladder cancer.

15. The method of claim 11, wherein said NK cell exosome preparation is administered more than once.

16. The method of claim 11, wherein said NK cell exosome preparation is administered on a chronic basis.

17. The method of claim 11, wherein said NK cell exosome preparation is administered systemically.

18. The method of claim 11, wherein said NK cell exosome preparation is administered intratumorally, or local or regional to a tumor.

19. The method of claim 11, wherein said cancer is metastatic, recurrent and/or multi-drug resistant.

20. The method of claim 11, wherein said NK cell exosome preparation is prepared using an NK cell from the subject, a healthy donor, umbilical cord blood or a NK cell line.

21. A method of treating a subject with an infectious disease comprising administering to said subject an NK cell exosome preparation prepared by the method of claim 1.

22-30. (canceled)
